(19) United States
(12) Patent Application Publication
(10) Pub. No.: US 2016/0166350 A1
(43) Pub. Date: Jun. 16, 2016

(54) Single-Use, Sterile Orthopedic Implant Kits and Methods of Providing Said Kits

(71) Applicant: FLOWER ORTHOPEDICS CORPORATION, Philadelphia, PA (US)

(72) Inventors: Oliver Burkhardt, Philadelphia, PA (US); Josef Zrinski, Wurmlingen (DE); Davor Zrinski, Rietheim-Weilheim (DE)

(73) Assignee: Flower Orthopedics Corporation, Philadelphia, PA (US)

(21) Appl. No.: 14/908,052

(22) PCT Filed: Jul. 31, 2014
(86) PCT No.: PCT/US14/49200

§ 371 Date: Jan. 27, 2016

Related U.S. Application Data
(60) Provisional application No. 61/861,519, filed on Aug. 2, 2013.

(51) Int. Cl. A61B 50/30; A61B 17/88; A61B 17/80; A61B 17/86; A61B 17/16; A61B 17/17

(52) U.S. Cl. CPC A61B 50/30; A61B 17/1615; A61B 17/17; A61B 17/80; A61B 17/86; A61B 17/8863; A61B 17/8875; A61B 2090/037

(57) ABSTRACT

Single-use, sterile orthopedic implant kits provide full carriages of kits comprising first individual sterile packages for a first surgical procedure at a first location, first individual sterile packages comprise one surgical component selected from sterile implant, sterile fasteners and disposable sterile surgical tool. The methods remove a portion of the first individual sterile packages from the full carriage to produce a used carriage, contents of the removed portion of the first individual sterile packages are usable to perform the first surgical. The methods return the used carriage to a provider at a second location for re-filling, to provide a refilled carriage of the kit by replacing the removed portion of the first individual sterile packages with second individual sterile packages having the same or contents of the removed portion of the first individual sterile packages, contents of the re-filled carriage are configured to be utilized during a second surgical procedure.

This application claims the benefit of U.S. Provisional Patent Application No. 61/861,519, filed Aug. 2, 2013, the entirety of which is hereby incorporated by reference into this application.

### FIELD OF THE DISCLOSURE

The present disclosure relates to single-use, sterile orthopedic implant kits comprising orthopedic implants, particularly bone plate implants, that are sterile packaged in kit containers along with the tools, appliances and/or fasteners that are needed, necessary or required to implant one or more of the orthopedic implants surgically into a patient. The bone plates, tools, appliances and fasteners comprise the kits and a plurality of components of the kits which are packaged in individual sterile containers and immediately ready for use upon unpackaging during surgical procedures without any pre-operation handling.

In an embodiment, a kit container contains one or more individually packaged sterile bone plates of various sizes. In an embodiment, the one or more individually packaged sterile bone plates may be of a particular type of bone plate, such as, for example, hand plates, foot plates, reconstruction plates, spine plates, stabilization nets or the like. The kit container also contains sterile packages of screws of various lengths and diameters. In an embodiment, the screws are of various sizes and are individually sterile packaged. In other embodiments, the kit may contain a first package of screws, containing a plurality of screws having the same diameter and/or same length, and a second package of screws that may contain one or more screws of the same size, but may be a different size from those in the first package of screws. Various other screw sizes can be included in the kit container and individually sterile packaged in the same manner.

Disposable tools and/or appliances are also included in individual sterile packages or multiple sterile packages in the kit container. In embodiments, the sterile disposable tools include, but are not limited to, a drill bit, bone plate bending tools, a bone plate template, a torque wrench, a drill guide, K-wires and/or a depth gauge. When a kit container contains screws having different diameters, one or more appropriately sized drill bits and/or drill guides may also be included in one or more sterile packages in the kit container. In embodiments, one or more of the sterile packages may each containing a drill bit and a corresponding or related drill guide.

During surgery, a surgeon will select the appropriately sized bone plate, optionally by using one or more sterile packaged disposable templates, and screws. Any of the disposable tools, appliances, templates that are used during the surgical procedure are subsequently discardable without any post-operation reprocessing. Subsequent to the surgical procedure, the remaining kit container will only contain sterile packages of components that were not used during the surgical procedure. The used kit container, with any remaining, unused, sterile packaged plates, screws, tools, appliances, trials and/or templates, is then sent back, returned and/or delivered to a provider for re-filling to replace the components that were used during the previous surgical procedure. As a result, a re-filled kit is provided, which is ready to be shipped to a subsequent surgery. In embodiments, the provider may be, for example, a producer, a supplier, a manufacturer, a distributor, a marketer or a combination thereof. The present disclosure should not be deemed as limited to a specific embodiment of the provider of the kit or the re-filled kit and the provider of the kit and the provider of the re-filled kit may be the same provider or different providers.

### SUMMARY OF THE DISCLOSURE

In embodiments, a single-use, sterile orthopedic implant kit may have a kit container containing at least one first individual sterile package, at least one second individual sterile package and at least one third individual sterile packages, wherein the at least one first individual sterile package contains at least one sterile implants, the at least one second individual sterile package contains at least one sterile fastener, and the at least one third individual sterile package contains at least one sterile surgical tool that is configured to be utilized during a surgical implant procedure and is disposable.

In an embodiment, the at least one sterile implant may be at least one sterile bone plate implant, the at least one sterile fastener may be at least one sterile screw, and the at least one sterile surgical tool may be a sterile drill bit that is disposable.

In an embodiment, the at least one sterile fastener may comprise at least one sterile locking screw and at least one sterile non-locking screw.

In an embodiment, the kit may have a carriage containing the first, second and third individual sterile packages, wherein the carriage is configured to be positioned inside of the kit container and is removable from the kit container.

In an embodiment, the carriage may have an organizer partitioning the carriage into a plurality of compartments, wherein the first, second and third individual sterile packages are positioned within the plurality of compartments.

In an embodiment, the kit may have a fourth individual sterile package comprising at least one selected from a sterile and disposable torque wrench, at least two sterile and disposable K-wires, a sterile and disposable depth gauge, and a sterile and disposable drill guide.

In an embodiment, the kit may have a fourth individual sterile package comprising a pair of sterile and disposable bone plate contouring tools, wherein each contouring tool has at least one channel sized to received and hold at least one portion of the bone plate implant for contouring or bending the bone plate implant.

In an embodiment, each contouring tool may have an overall shape that is cylindrical.

In an embodiment, the kit further may have a fourth individual sterile package comprising a sterile and disposable bone plate template corresponding to the bone plate implant, wherein the bone plate template is made of a frangible material such that the bone plate template is separable into two portions by hands of a user without aid of tools.

In embodiments, a method of may provide and re-fill a sterile orthopedic implant kit. The method may provide a full carriage of the kit comprising first individual sterile packages having contents configured to be utilized during a first surgical procedure at a first location, wherein each of the first individual sterile packages comprises one surgical component selected from at least one sterile implant, sterile fasteners and at least one disposable and sterile surgical tool. Further, the method may remove a portion of the first individual sterile packages from the full carriage of the kit to produce a used carriage of the kit, wherein contents of the removed portion of the first individual sterile packages are usable to perform or complete the first surgical procedure at the first location. Still further, the method may return the used carriage of the kit to a provider or a re-filling party at a second location for re-filing the used carriage. Moreover, the method may re-fill the used carriage to provide a re-filled carriage of the kit by replacing the removed portion of the first individual sterile packages with second individual sterile packages having the same or substantially the same contents as the contents of the removed portion of the first individual sterile packages, wherein contents of the re-filled carriage are configured to be utilized during a second surgical procedure.

In an embodiment, the at least one sterile implant may be a sterile bone plate implant, the sterile fasteners may be sterile screws, the at least one disposable and sterile tool may be a disposable and sterile drill bit, and the provider or the re-filling party may be a distributor or a manufacturer of the kit.

In an embodiment, the method may implant of a first portion of the contents of the removed portion of the first individual sterile packages during the first surgical procedure, and dispose of a second portion of the contents of removed portion of the first individual sterile packages during or after completion of the first surgical procedure.

In an embodiment, the method may deliver the re-filled carriage to a surgical suite or operating room prior to or during the second surgical procedure.

In an embodiment, the method may position the full carriage of the kit inside a kit container prior to delivering the full carriage of the kit to the first location.

In an embodiment, the sterile fasteners may comprise at least one sterile locking screw and at least one sterile non-locking screw.

In an embodiment, the at least one disposable and sterile surgical tool may further comprise at least one selected from a sterile and disposable torque wrench, at least two sterile and disposable K-wires, a sterile and disposable depth gauge, a sterile and disposable drill guide, a sterile and disposable drill bit.

In an embodiment, one of the first individual sterile packages may comprise a pair of sterile and disposable bone plate contouring tools, wherein each contouring tool has at least one channel sized to received and hold at least one portion of the bone plate implant for contouring or bending the bone plate implant.

In an embodiment, each contouring tool may have an overall shape that is cylindrical.

In an embodiment, one of the first individual sterile packages may comprise a sterile and disposable bone plate template corresponding to the bone plate implant, wherein the bone plate template is made of a frangible material such that the bone plate template is separable into two portions by hands of a user without aid of tools.

In an embodiment, the frangible material of the bone plate template may be aluminium.

### BRIEF DESCRIPTION OF THE DRAWINGS

So that the features and advantages of the present disclosure can be understood in detail, a more particular description of the systems and methods may be had by reference to the embodiments thereof that are illustrated in the appended drawings. It is to be noted, however, that the appended drawings illustrate only some typical embodiments of the present kits and methods and are therefore not to be considered limiting of its scope, for the kits and methods may admit to other equally effective embodiments.

FIG. 1 is a perspective view of a partially opened implant kit containing a carriage housing a plurality of individual sterile packages in an embodiment.

FIG. 2 is a perspective view of the carriage, shown in FIG. 1, having handled flaps and housing the plurality of individual sterile packages in an embodiment.

FIG. 3 is a perspective view of the carriage, shown in FIG. 2, having an organizer, with the plurality of individual sterile packages removed in an embodiment.

FIG. 4 is a top plan view of the carriage, shown in FIG. 3, in an embodiment.

FIG. 5 is a perspective view of an individual sterile package provided in the implant kit in an embodiment.

FIG. 6 illustrates a perspective view of an individual sterile package provided in the implant kit with contents of the individual sterile package outside of and alongside of the package in an embodiment.

FIG. 7 illustrates a perspective view of an individual sterile package with contents of the package outside of and alongside of the package in an embodiment.

FIG. 8 illustrates a perspective view of plate bending tools provided in the implant kit in an embodiment.

FIG. 9 illustrates a perspective view of an individual sterile package, containing one or more sterile bone plates or one or more sterile bone plate templates, provided in the implant kit in an embodiment.

FIG. 10A illustrates a top plan view of a sterile bone plate template provided, via an individual sterile package, in the implant kit in an embodiment; and FIG. 10B illustrates a top plan view of a sterile bone plate template, separated into portions, provided in the implant kit in an embodiment.

FIG. 11 illustrates a top plan view of a sterile bone plate implant provided, via an individual sterile package, in the implant kit in an embodiment.

FIG. 12 is a perspective view of a partially opened implant kit containing a carriage housing a plurality of individual sterile packages in an embodiment.

### DETAILED DESCRIPTION OF THE DISCLOSURE

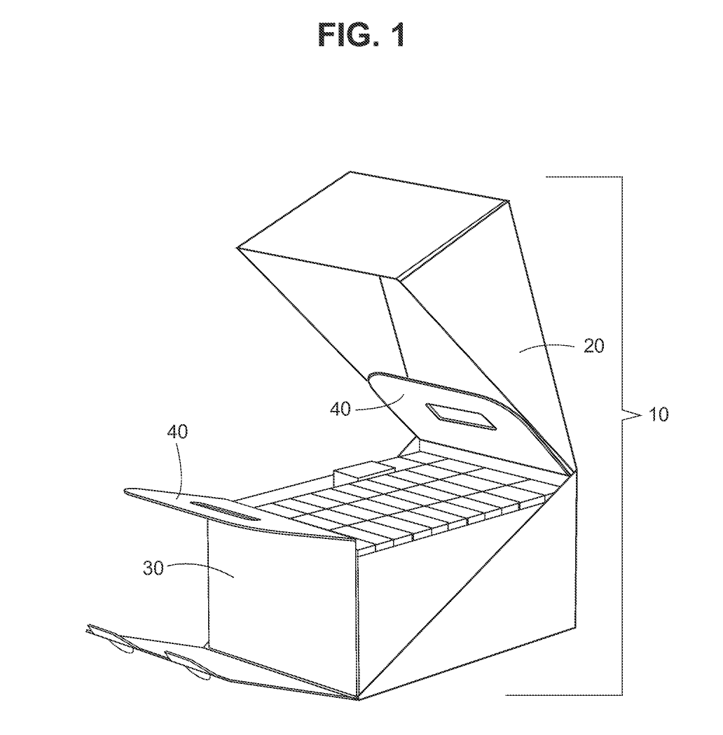

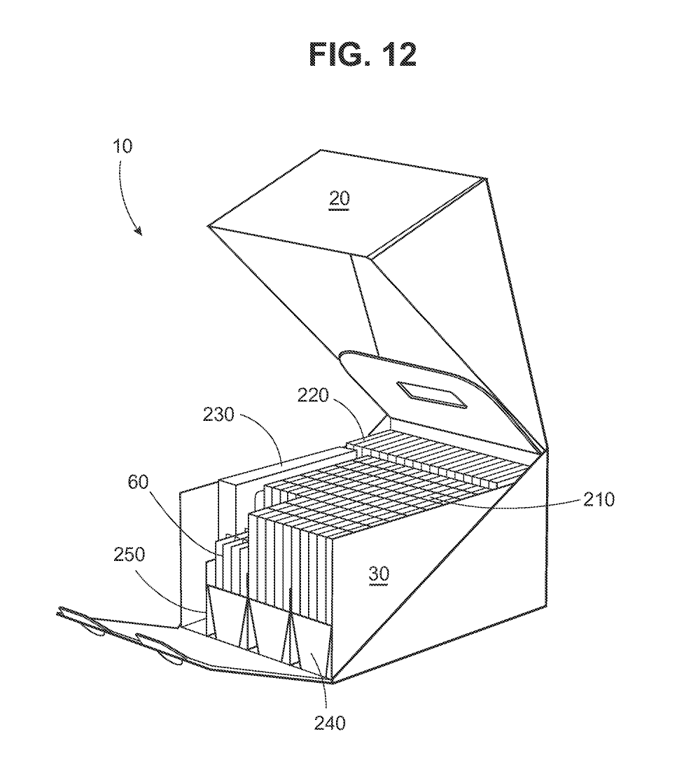

Referring now to the drawings wherein like numerals refer to like parts, a standardized, a single-use sterile orthopedic implant kit 10 (hereinafter “kit 10”) having an outer container 20 (hereinafter “container 20”) is provided at least partially opened as illustrated in FIGS. 1 and 12. The container 20 of the kit 10 is sized, shaped and/or configured to house, store, enclose and/or contain a removable carriage 30 (hereinafter “carriage 30). The carriage 30 is sized, shaped and/or configured to fit into and/or be housed within the container 20 and is easily removable from the container 20 via at least two handled flaps 40 located at two opposite sides and/or top edges of the carriage 30. The carriage 30 is preferably made from a coated paper or other protected or coated material so that carriage 30 and the contents of the carriage 30 may be separated from the container 20 and/or brought into an operating room or suite to be utilized during a surgical operation or procedure, such as, for example, a bone plate fixation procedure, an internal fixation procedure and/or a bone plating or implantation procedure (hereinafter “surgical procedure”).

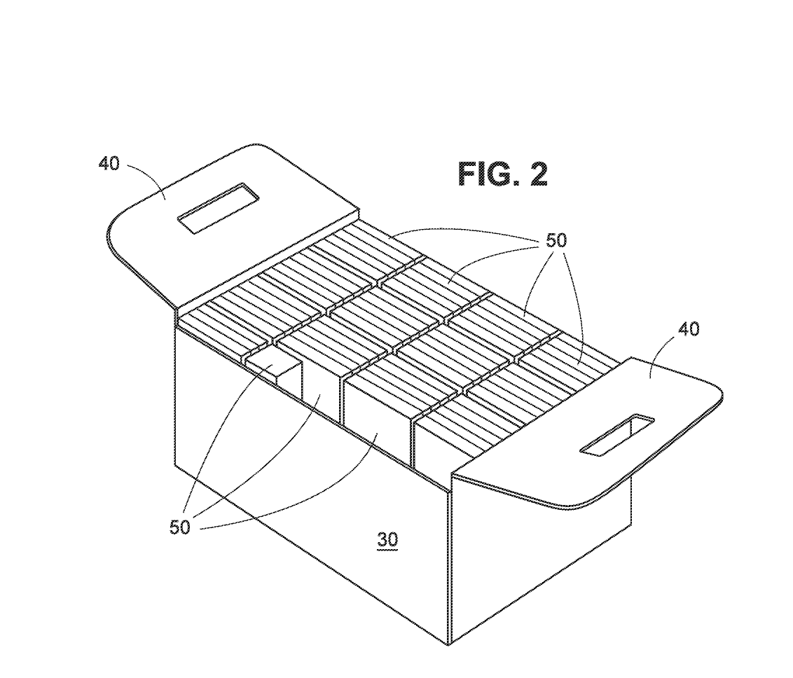

A plurality of individual sterile packages 50 (hereinafter “packages 50”), having sterile contents, are provided, housed, stored, arranged and/or positioned within the carriage 30 as shown in FIGS. 1, 2 and 12. The sterile contents of the packages 50 may include one or more of sterile surgical tools, sterile surgical appliances, sterile surgical screws, sterile surgical bone plate implants, sterile surgical bone plate templates, and/or other sterile surgical components necessary for completion of the surgical procedure. FIG. 2 also illustrates the carriage 30 having been removed or separated from the container 20 of the kit 10. In embodiments, the sterile tools, sterile appliances, sterile bone plate templates and/or other sterile components necessary for completion of surgical procedure are disposable and do not require any post-operation reprocessing after the completion of surgical procedure. In embodiments, the contents of the packages 50 are high-quality, sterile, single-use products that are immediately ready for use in the surgical procedure after unpackaging without any pre-operation handling.

The kit 10, the packages 50 and/or the contents of the packages 50 may be used for internal fixation of fractures and/or reconstruction of one or more bones. The one or more bones may include a scapula, olecranon, humerus, radius, ulna, pelvis, distal tibia, fibula, hand bones and/or foot bones. The internal fixation and/or reconstruction may include compression fractures, intra-articular and/or extra-articular factures, displaced fractures, osteotomies, non-unions and/or mal-unions. Moreover, the kit 10, the packages 50 and/or the contents of the packages 50 may be used for palmar, ventral, dorsal and/or orthogonal applications. It should be understood that the present disclosure is not limited to a specific embodiment of the one or more bones, the internal fixation, reconstruction and/or application for the kit 10, the packages 50 and/or the contents of the packages 50.

In embodiments, the contents of the packages 50 may include one of more of sterile bone plate implants, sterile bone plate templates, sterile and/or disposable surgical instruments or appliances, sterile wires, sterile drill bits, sterile locking screws, sterile non-locking screws, sterile locking systems, sterile bone plate bending tools or contouring systems and sterile reamers. The sterile wires may include, for example, K-wires and/or olive wires, and the sterile reamers may include a distal reamer and/or a reamer for a mediocarpal plate. One or more, or preferably all, of the contents and/or other components included in the kit 10 are housed, protected and/or stored within the individual sterile packages 50 such that contents of the kit 10 and the packages 50 are individually sterile packaged, easily openable or unpackaged, and immediately ready for utilization during the surgical procedure. At least some, if not most, of the contents of the kit 10 and/or the packages 50 may be single-use components which are disposable after being used during the surgical procedure. The type or configuration of the contents of the kit 10 and/or the packages 50 are determined, identified and/or selected based on the type of surgery procedure that is necessary or needed by the patient. For example, different kits containers include different contents based on the different types of bone-fixation or implantation surgeries that are necessary or needed by different patients. Each kit 10 is tailored to the specific surgical procedure (i.e., hand, wrist ankle, etc.) and the contents of kit 10 and/or the packages 50 may include one or more sterile bone plate implants, one or more sterile screws, one or more sterile bone plate templates and/or one or more sterile disposable surgical instruments and/or appliances needed by a surgeon to perform a specific surgical procedure. For example, the kit 10 may contain (i) different sterile packaged bone plate implants that the surgeon needs for the specific surgical procedure, (ii) sterile packaged screws, (iii) sterile packaged disposable instruments and/or appliances, (iv) sterile packaged disposable drill bits and/or (v) sterile packaged disposable bone plate templates.

In embodiments, the contents of the kit 10 and/or of the packages 50 may be immediately ready for surgery after unpackaging, are sterile packaged, require no pre-operation or post-operation reprocessing, eliminate reprocessing costs, limit risk of infection and expedite operating room or suite turnaround. In an embodiment, the standardized bone-fixation provided by the contents of the kit 10 and/or the packages 50 may provide (i) at least one sterile locking mechanism for the bone plate implant, (ii) a sterile and disposable instrument kit, (iii) a reduced inventory for the surgeon or surgical facility and/or (iv) no need for in-surgery representative support during the surgical procedure.

The individual sterile packages 50 that house, store and protect the contents of the kit 10 may comprise clear sterile-packaging so that the contents of each package 50 is clearly visible to allow for quick and easy identification of the contents of each package 50 during the surgical procedure and/or during re-filling by the provider. Additionally, the packages 50 may, in embodiments, display markings or indicia and/or have colour coding which may be indicative of the contents of each package 50. As a result, the markings, indicia and/or colour coding may simplify handling and provide easy recognition of the contents of each package 50 during the surgical procedure and/or during re-filling by the provider. In embodiments, each of the packages 50 may contain, have or display, for example, one or more scanable images to facilitate quick, easy and reliable identification of the contents of the packages along with facilitating automated tracking, ordering and/or re-ordering or re-filling of specific contents of the kit 10 when such specific contents of the kit 10 have been depleted or utilized during the surgical procedure. In an embodiment, each of the packages 50 may have one or more bar codes and/or quick response codes that are indicative of the contents of each package 50. The present disclosure should not be deemed limited to a specific embodiment of markings, indicia, colour coding and/or one or more scanable images displayable on each of the packages 50.

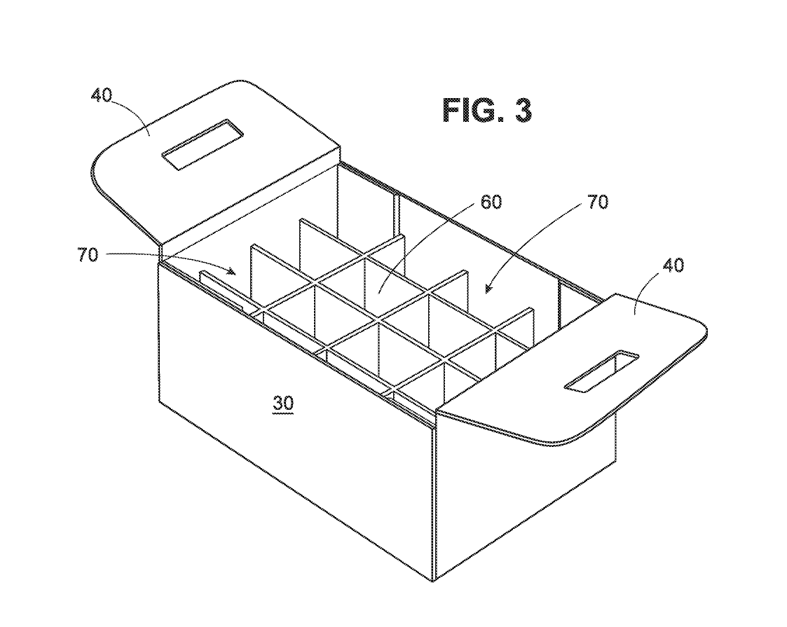

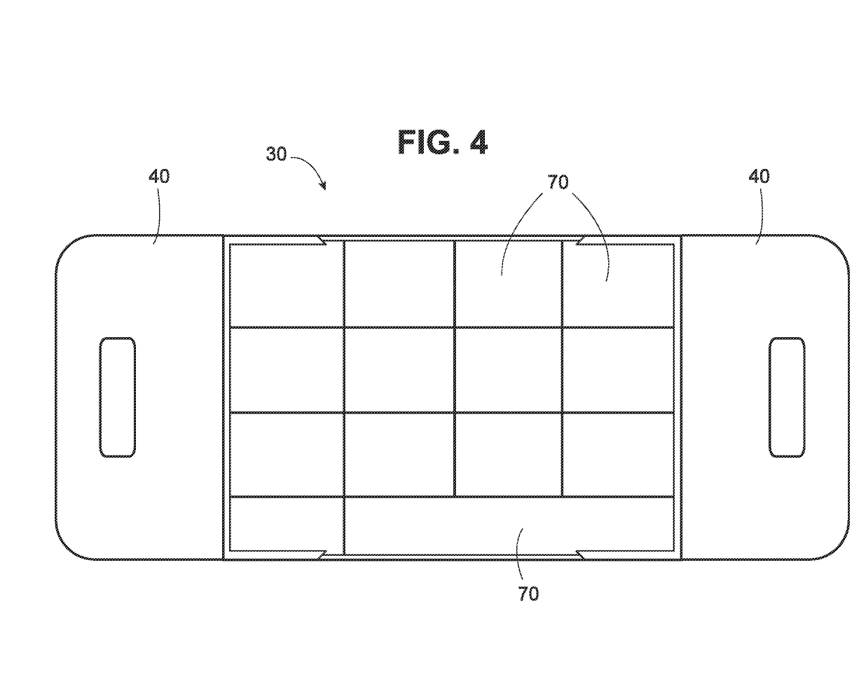

FIG. 3 illustrates the carriage 30 of the kit 10, in an empty state or condition, that comprises an organizer 60 positioned or provided within the interior of the carriage 30. The organizer 60 partitions, separates and/or divides the interior of the carriage 30 into a plurality of compartments 70 (hereinafter “compartments 70”) as shown in FIGS. 3, 4 and 12. In embodiments, the organizer 60 may be removable from the carriage 30 and/or may be made of one or more coated paper materials and/or other coated materials. The compartments 70 are sized, shaped and/or configured to receive, house, store and/or organize the packages 50 provided in the kit 10. For example, a first compartment 70 may be configured to receive, house and/or store one or more first packages 50 that contain sterile fasteners or screws and sterile tool kits while remaining second compartments 70 may be used to receive, house and/or store one or more second packages 50 containing other sterile surgical components necessary for completing the surgical procedure, such as, for example, at least one sterile bone plate and/or sterile bone plate template. It should be understood that the present disclosure should not be deemed as limited to a specific embodiment of the coated material of the carriage 30.

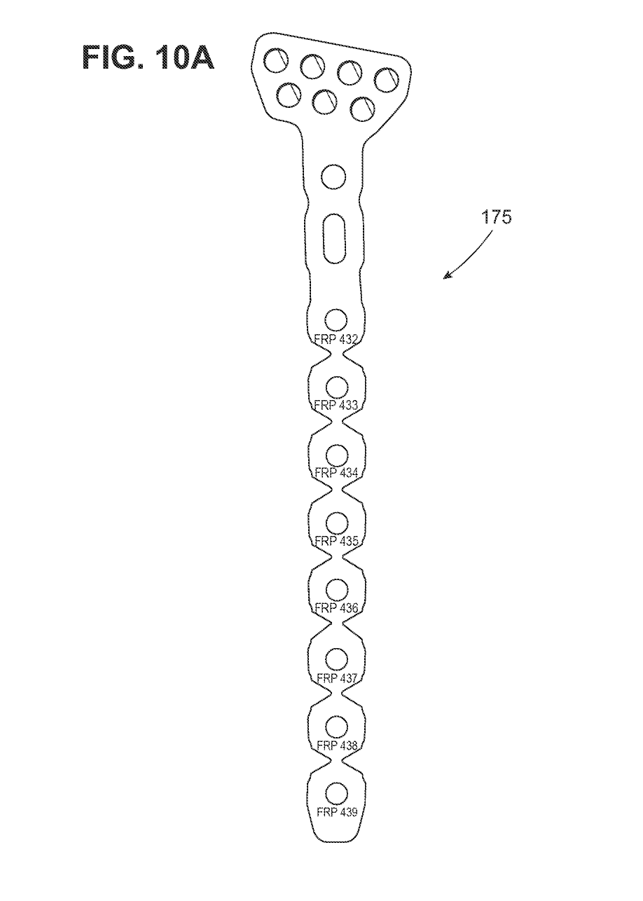

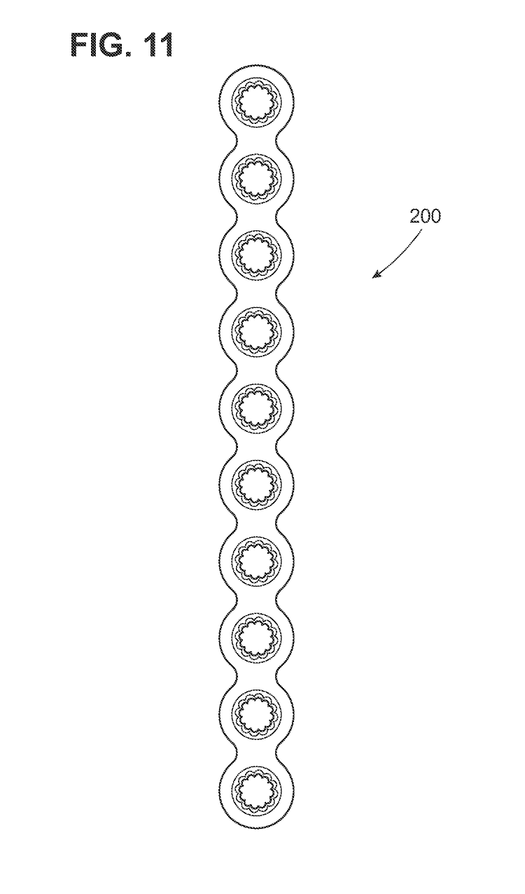

In embodiments, at least one package 50 provided in the kit 10 comprises at least one sterile packaged bone plate implant, such as, for example, an anatomic plate, a reconstruction plate or an osteosynthesis plate. For example, an individual sterile package 170 as shown in FIG. 9 may contain at least one sterile bone plate implant 200 (hereinafter “bone plate implant 200”) as shown in FIG. 11 and/or a corresponding bone plate template. In an embodiment, the at least one bone plate implant may be, for example, at least one veterinary orthopedic implant. Outer surfaces of the at least one bone plate implant may be made of one or more biomedical materials, such as, for example, titanium, silicone, apatite and/or the like. The at least one bone plate implant or bone plate implant 200 may be affixed, attached, connected and/or secured to the bone during the surgical procedure via one or more sterile fasteners and/or screws. In embodiments, the one or more sterile fasteners and/or screws may comprise one or more sterile locking screws, one or more sterile non-locking screws or a combination of sterile locking and sterile non-locking screws. The one or more sterile screws may be fully threaded screws or at least partially threaded screws. Each sterile screw is sized, shaped and/or configured to be received by a hole formed in each bone plate implant for fastening, securing and/or attaching the bone plate implant to the bone during the surgical procedure. One or more sterile washers may be provided in at least one of the packages 50 and may be utilized during the surgical procedure to affix, connect, attach or secure the at least one bone plate to the bone during the surgical procedure.

In embodiments, the at least one bone plate implant or the bone plate implant 200 may be one selected from the group consisting of a hand plate, a foot plate, a reconstruction S-plate, a reconstruction M-plate, a proximal humerus plate, a distal radius plate and an osteosynthesis S-plate. In an embodiment, the hand and/or foot plate may be, for example, a straight plate, a L-plate, an oblique T-plate, a T-plate, a H-plate, a mediocarpal plate, an elongated L-plate, a straight plate with slots, or an angled and/or inclined plate. In an embodiment, the reconstruction S-plate may be, for example, a straight plate or a T-plate. In an embodiment, the distal radius plate may be, for example, volar, narrow L-plate, a volar, narrow R-plate, a volar, wide L-plate, a volar, wide R-plate, a dorsal L-plate, a dorsal R-plate, a volar L-plate or a volar R-plate. The at least one bone plate implant or the bone plate implant 200 may have a thickness, such as, for example, less than about 4.0 millimetres (hereinafter “mm”), less than about 3.0 mm, less than about 2.0 mm, less than about 1.5 mm or about 1.0 mm. It should be understood that the present disclosure is not limited to a specific embodiment of the at least one bone plate implant, the one or more biomedical materials on the outer surfaces of the implant and/or the thickness of the implant. The at least one bone plate implant or the bone plate implant 200 may be any bone plate implant utilized during the surgical procedure as known to one of ordinary skill in the art.

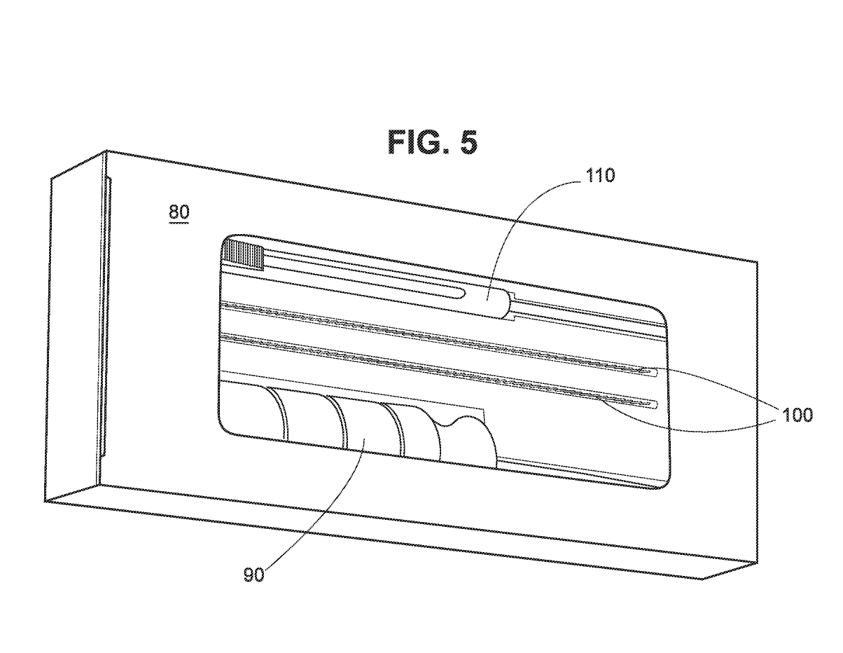

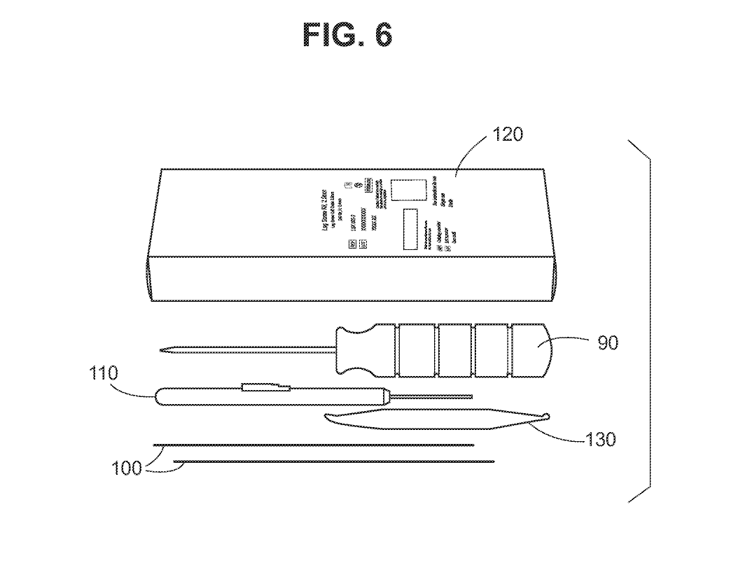

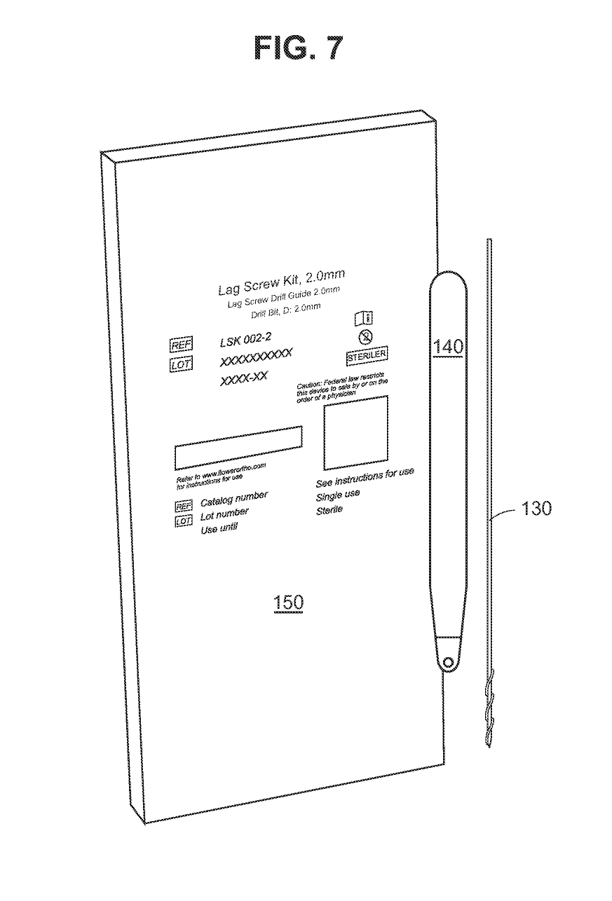

At least one of the packages 50 provided in the kit 10 and/or the carriage 30 comprises a tool kit. Each tool kit provided in the kit 10 and/or contained within at least one of the packages 50 may include implant-based sterile surgical instruments, appliances and/or tools, needed for completing the surgical procedure, that are sterile, robust and/or disposable. Each tool kit provided in the kit 10 may add assurance that the correct surgery-specific tools are present at the surgical room or suite during the surgical procedure and are in optimal or prime condition for completing the surgical procedure. In embodiments, at least one of the packages 50 may be an individual sterile tool kit package, such as, for example, a sterile K-wire kit package 80 as shown in FIG. 5, a sterile olive wire kit package, a sterile guide wire kit package, a sterile drill bit kit package, a sterile lag screw kit package 150 as shown in FIG. 7, a sterile cannulated drill bit kit package, a sterile bone plate implant and/or template kit package 170 as shown in FIG. 9, a sterile bone plate benders kit package, a sterile mediocarpal reamer kit package, a sterile cannulated reamers kit package, a sterile cannulated countersinks kit package and/or a sterile hybrid or combination tool kit package 120 as shown in FIG. 6.

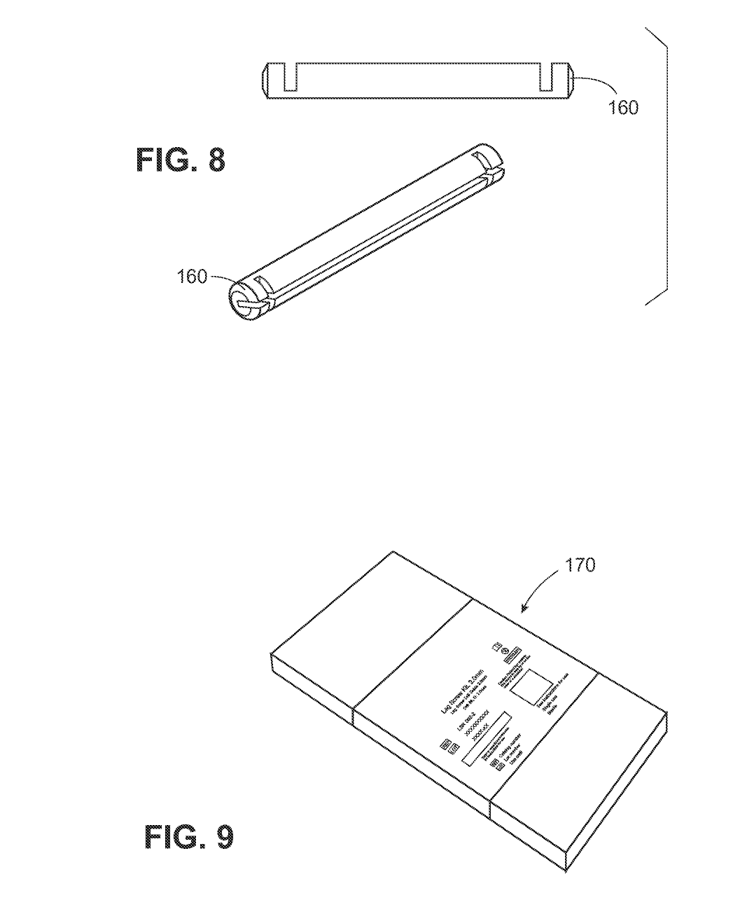

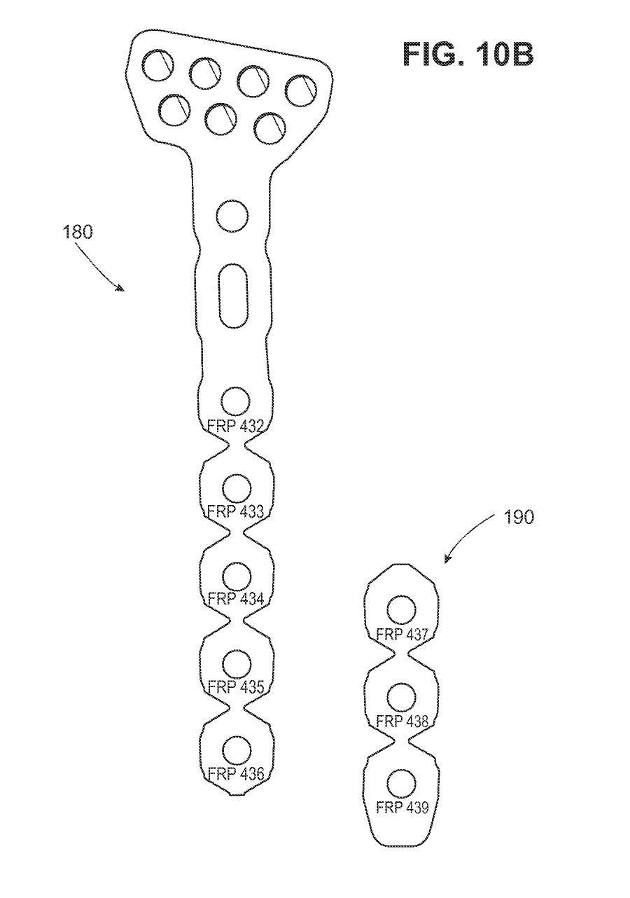

In an embodiment, the sterile K-wire kit package 80, shown in FIG. 5, may comprise at least one of a sterile and disposable torque wrench 90, at least two sterile and disposable K-wires 100 and/or a sterile and disposable depth gauge 110. In an embodiment, sterile hybrid or combination tool kit package 120, shown in FIG. 6, may comprise at least one of the sterile and disposable torque wrench 90, at least two sterile and disposable K-wires 100, a sterile and disposable depth gauge 110 and/or a sterile and disposable drill guide 130. In an embodiment, the sterile lag screw kit package 150, shown in FIG. 7, may comprise at least one of the sterile and disposable drill guide 130 and a sterile and/or disposable drill bit 140. In an embodiment, the sterile bone plate benders kit package (not shown in the drawings) may comprise a pair of sterile and disposable bone plate bending or contouring tools 160 (hereinafter “plate bending tools 160”), as shown in FIG. 8, which may be utilized to bend, shape and/or contour a bone plate implant, such as, for example, the bone plate implant 200 as shown in FIG. 11. In an embodiment, the sterile bone plate implant and/or template kit package 170, shown in FIG. 9, may comprise at least one of the bone plate implant 200, shown in FIG. 11, and/or at least one sterile and disposable bone plate template 175, shown in FIG. 10A, which may be torn, divided and/or separated by hand, without the aid of an additional tool, into at least a first portion 180 and a second portion 190 as shown in FIG. 10B.

In embodiments, one or more portions of the plate bending tools 160, shown in FIG. 8, may be shaped, sized and/or configured to receive, hold, contour, shape and/or manipulate at least one bone plate implant (i.e., the bone plate implant 200) prior to affixing the at least one bone plate implant to the bone during the surgical procedure. Bone plate implants frequently require bending, twisting, contouring and/or shaping in an operating room or suite so that the medical implants may have a contour and/or shape that correspond to a contour and/or shape of the bone before being affixed to said bone. The present kit 10 may provide inexpensive, sterile packaged, disposable plate bending tools for easily receiving, holding, bending, twisting, manipulating, contouring and/or shaping one or more bone plate implants. It should be understood that contouring the bone plate implant may also mean or include angling the implant, bending the implant, twisting the implant, turning the implant, manipulating the implant, and/or shaping the implant.

In an embodiment, the plate bending tools 160 may have an overall shape that is cylindrical or polygonal. In embodiment, the plate bending tools 160 may have a circular or oval cross-sectional shape that may extend along a portion of the length, or along the entire length, of the plate bending tools 160. The length of the plate bending tools 160 may be defined between a first end and a second end located opposite to the first end of the plate bending tools 160. Each of the plate bending tools 160 may have a first channel extending across a portion of the length, or along the entire length, of the plate bending tools 160. Each of the plate bending tools 160 may have one or more second channels extending across a width of the plate bending tools 160 such that the one or more second channels may intersect, overlap, contact or abut at least one portion of the first channel of each plate bending tool 160. For example, the plate bending tools 160 may have two second channels whereby one second channel is located adjacent to the first end of the plate bending tools 160 and the other second channel is located adjacent to the second end of the plate bending tools 160. As a result, the second channels may intersect, overlap, contact or abut at least two portions of the first channel of each plate bending tool 160. In an embodiment, the one or more second channels may extend perpendicularly with respect to the first channel of each plate bending tool 160.

In embodiments, the first and/or second channels of the plate bending tools 160 may be sized, shaped and/or configured to receive and/or hold portions of the bone plate implant such that a user (i.e., surgeon, surgical staff member, medical assistant or provider, etc.) may manipulate, move or use the plate bending tools 160 to manipulate contour, angle, bend, twist, turn and/or shape one or more portions of the bone plate implant. As a result, the bone plate implant may be contoured, via the plate bending tools 160, to have a shape or contour that corresponds to the shape or contour of the bone in which the bone plate implant is subsequently being affixed or connected to. After the bone plate implant is contoured, bent or shaped, by the plate bending tools 160, the plate bending tools 160 may be disposed of without post-operation reprocessing.

In embodiments, the at least one sterile and/or disposable bone plate template 175 (hereinafter “template 175”) is sterile-packaged and available for all surgical implant procedures or applications and may be used to pinpoint the exact implant size prior to opening the package 50 containing the necessary and correct bone plant implant for the surgical procedure. In an embodiment, the template 175 may have laser-marked numbering or indicia on at least the second portion 190, which is hand removable from the first portion 180, indicating or corresponding to the correct implant size for the surgical procedure. Each template 175 has at least one removable segment (i.e., the second portion 190) that may be torn, removed or separated, by hand and without the aid of an additional tool, to determine, indicate and/or identify the correct, suitable or appropriate size, shape and/or length of the bone plate implant to be affixed to the bone during the surgical procedure. At least one side of the template 175 may have or display indicia which may be used to determine or identify the correct, suitable and/or appropriate bone plate implant to be utilized during the surgical procedure. Each template 175 and corresponding bone plate implant may be individually sterilized and individually packaged in one or more packages 50 such that each template 175 and the bone plate implants are immediately ready for use in the surgical procedure after unpackaging without any pre-operation processing. After the bone plate implant having the necessary and correct size, shape and/or length for the surgical procedure is determined or identified based on the second portion 190 of the template 175, the first and second portions 180, 190 of the template 175 are disposed of without any post-operation processing.

In embodiments, each template 175 may be one selected from the group consisting of a mediocarpal plate template, a recon plate template, a H & F plate template, a recon T-plate template, a straight osteo plate template, a H-plate template, a dorsal distal radius plate template, volar distal radius plate template, a narrow volar distal radius plate template, a wide volar distal radius plate template, a humerus plate template, a straight osteo plate template, an elongated L-plate template, an angled & inclined plate template, and a straight plate with slots template. The templates may have one or more structural features that correspond to one or more structural features of the bone plate implants necessary for the surgical procedure. In embodiments, the templates may have outer perimeters, curvatures, indentations, angles, ridges and/or holes which may correspond to outer perimeters, curvatures, indentations, angles, ridges and/or screw holes of the bone plate implants included in the kit 10, which are necessary for the surgical procedure.

In embodiments, the template 175 is made of at least one frangible material such that the template 175 may be torn, broken or separated into at least first and second portions 180 and 190 without the use of a tool, by hands of the user. In embodiments, the second portion 190 of the template 175 may be made of the frangible material and the first portion may 180 be made of a non-frangible material such that the second portion 190 of the template 175 may be torn, fragmented, broken or separated away from the first portion 180, without the use of a tool, by hands of the user. The at least one frangible material may include, for example, one or more frangible metal materials, one or more frangible polymer materials, one or more frangible composite materials and/or combinations thereof. In an embodiment, the frangible material may be a frangible metal material, such as, for example, aluminium.

FIG. 12 shows the carriage 30 housed within the container 20 of the kit 10, wherein the plurality of individual sterile packages 50 comprises one or more individual sterile screw boxes 210 (hereinafter “screw boxes 210”), one or more individual sterile implant and/or appliance boxes 220 (hereinafter “implant/appliance boxes 220”), and/or one or more individual sterile instrument kit boxes 230 (hereinafter “instrument kit boxes 230”). The screw boxes 210, the implant/appliance boxes 220 and/or the instrument kit boxes 230 may be positioned, provided and/or located within the compartments 70 formed or provided by the organizer 60 as shown in FIG. 12.

A highground or gate 240 (hereinafter “gate 240”) may be provided at a top side of a floor of the carriage 30 and at a front side of the carriage 30. The front side of the carriage 30 is located opposite with respect to the back side of the carriage 30 and, when the container 20 is at least partially open, a back side of the carriage 30 remains covered or concealed by at least one wall of the container 20 and the front side of carriage 30 is opened, exposed and accessible. The gate 240 at the front side of the carriage 30 extends upward and/or away from the top side of the floor of the carriage 30 such that the gate will prevent or restrict forward movement of the contents of the carriage 30 (i.e., screw boxes 210, implant/appliance boxes 220 and/or instrument kit boxes 230). Thus, the gate 240 may prevent or restrict the contents of the carriage 30 from accidently falling out of the carriage 30 and container 20 when the container 20 is at least partially opened. As a result, the gate 240 may prevent or reduce accidental damage to the contents of the carriage 30. The carriage 30 may also comprise at least one isolator or petition 250 (hereinafter “petition 250”) which may be sized, shaped and/or configured to further divide or separate the compartments 70 formed by the organizer 60. Said further division or separation is achieved by the petition 250 extending upward and/or away from the top side of the floor of the carriage 30. The gate 240 and/or the petition 250 may be made of a coated paper material, a coated polymer material, or a combination thereof. It should be understood that the present disclosure is not limited to a specific embodiment of the coated material(s) of the gate 240 and/or the petition 250.

In embodiments, the screw boxes 210 may comprise at least one selected from sterile fasteners, sterile non-locking screws, sterile locking screws, sterile cannulated screws (partially threaded, fully threaded or a combination thereof), and/or sterile washers. Further, the implant/appliance boxes 220 may comprise at least one selected from sterile bone plate implants (i.e., bone plate implant 200), sterile bone plant templates, sterile K-wire kits, sterile olive wire kit, sterile distal reamers, sterile bone plate benders, sterile guide wire kits, and/or sterile cannulated countersinks. Moreover, the instrument kit boxes 230 may comprise at least one selected from drill bit kits, lag screw kits, and/or cannulated drill bits.

In embodiments, a provider (i.e., producer, supplier, manufacturer, distributor, marketer, etc.) may prepare the kit 10 by including, positioning or providing first individual sterile packages 50 that are necessary for completing a first surgical procedure within the carriage 30 of the kit 10. The kit 10, having the first packages 50, is provided to a user (i.e., surgeon, surgical staff member, medical assistant or provider, etc.) at a first location for utilization before and/or during the first surgical procedure. Subsequently, one or more of the first packages 50 are removed from the carriage 30 for utilization before and/or during the first surgical procedure. The one or more packages 50 removed from the carriage 30 may contain the necessary and/or correct sterile- and individually-packaged surgical components (i.e., bone plate implant, fasteners, bone plate template, drill bit, bone plate benders, etc.) for completing the first surgical procedure.

The one or more packages 50 removed from the carriage 30 may be opened at a same time or at different times before and/or during the first surgical procedure and the contents of the one or more packages 50 may be removed. A first portion of the contents removed from the one or more packages 50 (i.e., bone plate implant, fasteners, washers, etc.) may be utilized and/or implanted into the patient during the first surgical procedure without any pre-operation processing. A second portion of the contents removed from the one or more packages 50 (i.e., bone plate template, drill bit, bone plate benders, etc.) are disposable and, therefore, may be disposed during the first surgical procedure or after completion of the first surgical procedure without any post-operation reprocessing.

After the first surgical procedure has been completed, the used kit 10, containing the remaining first packages 50 that were not utilized during first surgical procedure, is delivered or returned to the provider or a re-filling party at a second location which may be local or remote with respect to the first location. At the second location, the provider or the re-filling party may refill or reload the kit 10 so that the kit 10 once again contains all the necessary and/or correct sterile- and individually-packaged surgical components (i.e., bone plate implant, fasteners, bone plate template, drill bit, bone plate benders, etc.) for completing a second surgical procedure at the first location or a third location that may be local or remote with respect to the first location. The first and second surgical procedures may be the same type of surgical procedures or different types of surgical procedures.

To refill or reload the kit 10, the provider or re-filling party determines which first packages 50 were removed from the carrier 30 and/or utilized to complete the first surgical procedure. After determining the first packages 50 that were removed from the carrier 30, the provider or re-filling party replaces, refills and/or reloads the kit 10 with new or replacement individual sterile second packages 50 that have the same or replacement contents as the original first packages 50 that were removed from the carrier 30 during the first surgical procedure. As a result, the kit 10 is refilled or reloaded, contains both original first packages 50 and newly added or replacement second packages 50, and provides all the necessary and/or correct sterile- and individually-packaged surgical components (i.e., bone plate implant, fasteners, bone plate template, drill bit, bone plate benders, etc.) for completing the second surgical procedure at the first location or the third location. After the kit 10 has been refilled, the refilled kit 10 may be sent, delivered or transported from the second location to the first location or the third location to be utilized before and/or during the second surgical procedure.

Upon completion of the second surgical procedure, the kit 10, excluding the original and previously added packages 50 that were removed from the carriage 30 before and/or during the second surgical procedure, may be returned or delivered to the provider or the re-filling party at the second location. Once again, the kit 10 may be refilled or restocked with new packages 50 that replace the packages 50 utilized during the second surgical procedure. As a result, the kit 10 may again be refilled with all the necessary and/or correct sterile- and individually-packaged surgical components (i.e., bone plate implant, fasteners, bone plate template, drill bit, bone plate benders, etc.) for completing a third surgical procedure. The kit 10 may be refilled any number of times and one or more of the packages 50 may be refilled or replaced any number of times such that the refillable kit 10 may contain all the necessary and/or correct sterile- and individually-packaged surgical components to complete any number of surgical procedures.

It will be appreciated that several of the above-disclosed and other features and functions, or alternatives thereof, may be desirably combined into many other different kits and/or methods. Also, various presently unforeseen or unanticipated alternatives, modifications, variations or improvements therein may be subsequently made by those skilled in the art, and are also intended to be encompassed by the present disclosure.

We claim:

1. A single-use, sterile orthopedic implant kit comprising:
a kit container containing at least one first individual sterile package, at least one second individual sterile package and at least one third individual sterile packages,
wherein the at least one first individual sterile package contains at least one sterile implants, the at least one second individual sterile package contains at least one sterile fastener, and the at least one third individual sterile package contains at least one sterile surgical tool that is configured to be utilized during a surgical implant procedure and is disposable.

2. The kit according to claim 1, wherein the at least one sterile implant is at least one sterile bone plate implant, the at least one sterile fastener are at least one sterile screw, and the at least one sterile surgical tool is a sterile drill bit that is disposable.

3. The kit according to claim 1, wherein the at least one sterile fastener comprises at least one sterile locking screw and at least one sterile non-locking screw.

4. The kit according to claim 1, the kit further comprising:
a carriage containing the first, second and third individual sterile packages, wherein the carriage is configured to be positioned inside of the kit container and is removable from the kit container.

5. The kit according to claim 1, the carriage further comprising:
an organizer partitioning the carriage into a plurality of compartments, wherein the first, second and third individual sterile packages are positioned within the plurality of compartments.

6. The kit according to claim 2, the kit further comprising:
a fourth individual sterile package comprising at least one selected from a sterile and disposable torque wrench, at least two sterile and disposable K-wires, a sterile and disposable depth gauge, and a sterile and disposable drill guide.

7. The kit according to claim 2, the kit further comprising:
a fourth individual sterile package comprising a pair of sterile and disposable bone plate contouring tools, wherein each contouring tool has at least one channel sized to received and hold at least one portion of the bone plate implant for contouring or bending the bone plate implant.

8. The kit according to claim 7, wherein each contouring tool has an overall shape that is cylindrical.

9. The kit according to claim 2, the kit further comprising:
a fourth individual sterile package comprising a sterile and disposable bone plate template corresponding to the bone plate implant, wherein the bone plate template is made of a frangible material such that the bone plate template is separable into two portions by hands of a user without aid of tools.

10. A method of providing and re-filling a sterile orthopedic implant kit, the method comprising:
providing a full carriage of the kit comprising first individual sterile packages having contents configured to be utilized during a first surgical procedure at a first location, wherein each of the first individual sterile packages comprises one surgical component selected from at least one sterile implant, sterile fasteners and at least one disposable and sterile surgical tool;
removing a portion of the first individual sterile packages from the full carriage of the kit to produce a used carriage of the kit, wherein contents of the removed portion of the first individual sterile packages are usable to perform or complete the first surgical procedure at the first location;
returning the used carriage of the kit to a provider or a re-filling party at a second location for re-filing the used carriage; and
re-filling the used carriage to provide a re-filled carriage of the kit by replacing the removed portion of the first individual sterile packages with second individual sterile packages having the same or substantially the same contents as the contents of the removed portion of the first individual sterile packages, wherein contents of the re-filled carriage are configured to be utilized during a second surgical procedure.

11. The method according to claim 10, wherein the at least one sterile implant is a sterile bone plate implant, the sterile fasteners are sterile screws, the at least one disposable and sterile tool is a disposable and sterile drill bit, and the provider or the re-filling party is a distributor or a manufacturer of the kit.

12. The method according to claim 11, further comprising:
implanting of a first portion of the contents of the removed portion of the first individual sterile packages during the first surgical procedure; and
disposing of a second portion of the contents of removed portion of the first individual sterile packages during or after completion of the first surgical procedure.

13. The method according claim 10, further comprising:
delivering the re-filled carriage to a surgical suite or operating room prior to or during the second surgical procedure.

14. The method according to claim 10, further comprising:
positioning the full carriage of the kit inside a kit container prior to delivering the full carriage of the kit to the first location.

15. The method according to claim 10, wherein the sterile fasteners comprise at least one sterile locking screw and at least one sterile non-locking screw.

16. The method according to claim 10, wherein the at least one disposable and sterile surgical tool further comprises at least one selected from a sterile and disposable torque wrench, at least two sterile and disposable K-wires, a sterile and disposable depth gauge, a sterile and disposable drill guide, a sterile and disposable drill bit.

17. The method according to claim 11, one of the first individual sterile packages comprises a pair of sterile and disposable bone plate contouring tools, wherein each contouring tool has at least one channel sized to received and hold at least one portion of the bone plate implant for contouring or bending the bone plate implant.

18. The method according to claim 17, wherein each contouring tool has an overall shape that is cylindrical.

19. The method according to claim 11, one of the first individual sterile packages comprises a sterile and disposable bone plate template corresponding to the bone plate implant, wherein the bone plate template is made of a frangible material such that the bone plate template is separable into two portions by hands of a user without aid of tools.

20. The method according to claim 19, wherein the frangible material of the bone plate template is aluminum.
